(19) United States
(12) Patent Application Publication
(10) Pub. No.: US 2020/0063098 A1
(43) Pub. Date: Feb. 27, 2020

(54) USE OF N-BUTYLIDENEPHTHALIDE IN DOPAMINERGIC PROGENITOR CELL TRANSPLANTATION

(71) Applicant: ULTRA-MICRORIGIN BIOMEDICAL TECHNOLOGY CO., LTD., Hsinchu City (TW)

(72) Inventors: Ming-Hsi CHUANG, Hsinchu City (TW); Lin-Hsiang CHUANG, Hsinchu City (TW); Po-Cheng LIN, Hsinchu City (TW); Chia-Hsin LEE, Hsinchu City (TW); Yi-Chun LIN, Hsinchu City (TW); Chi-Hsuan CHUANG, Hsinchu City (TW); Shinn-Zong LIN, Hsinchu City (TW); Chia-Yu CHANG, Hsinchu City (TW); Ching-I SHEN, Hsinchu City (TW)

(21) Appl. No.: 16/184,708

(22) Filed: Nov. 8, 2018

(30) Foreign Application Priority Data
Aug. 23, 2018 (TW) 107129513

(51) Int. Cl. C12N 5/0793; A61K 35/30; A61K 31/365

(52) U.S. Cl. CPC C12N 5/0619; A61K 35/30; C12N 2506/08; C12N 2501/115; C12N 2501/999; A61K 31/365

(57) ABSTRACT

Uses of n-butylidenephthalide (BP) in dopaminergic progenitor cell transplantation are provided, wherein the uses include using BP to enhance the therapeutic effect of dopaminergic progenitor cell transplantation, and using a combination of BP and BP-treated dopaminergic progenitor cells in dopaminergic progenitor cell transplantation. The uses especially relate to using BP to enhance the therapeutic effect of dopaminergic progenitor cell transplantation on Parkinson's disease.

### CROSS REFERENCE TO RELATED APPLICATIONS

This application claims priority to Taiwan Patent Application No. 107129513 filed on Aug. 23, 2018, in the Taiwan Intellectual Property Office, the disclosure of which is incorporated herein in its entirety by reference.

### FIELD OF THE INVENTION

The present invention relates to uses of n-butylidenephthalide (BP) in cell transplantation, especially in dopaminergic progenitor cell transplantation. The uses include using BP to enhance the therapeutic effect of dopaminergic progenitor cell transplantation, and using a combination of BP and BP-treated dopaminergic progenitor cells in dopaminergic progenitor cell transplantation. The uses especially relate to using BP to enhance the therapeutic effect of dopaminergic progenitor cell transplantation on Parkinson's disease.

### BACKGROUND OF THE INVENTION

The cause of Parkinson's disease, a common degenerative disease of the central nervous system, primarily resides in the decreased dopamine secretion due to the degeneration and/or death of dopaminergic neurons. Patients with Parkinson's disease will lose their motor control ability gradually and have difficulty in body movements. Currently, drugs for clinical use in treating Parkinson's disease (e.g., L-dopa) control the condition of patients by increasing the amount of dopamine in the patients' body. However, when the death of dopaminergic neurons in a patient reaches a certain level along with the progression of disease, the therapeutic effect that can be provided by L-dopa or a more invasive stimulation treatment is rather limited.

Therefore, both the industries and research institutes have been devoting in the development of a drug or method for treating Parkinson's disease effectively. In recent years, dopaminergic progenitor cell transplantation has brought new opportunities in treating Parkinson's disease. The term “dopaminergic progenitor cell transplantation” refers to transplanting dopaminergic progenitor cells to a patient's brain, and thus, the transplanted dopaminergic progenitor cells will differentiate into dopaminergic neurons, thereby increasing the number of dopaminergic neurons in the patient and promoting the neurite outgrowth therein. However, researchers have discovered that though dopaminergic progenitor cells transplanted as neurospheres to a patient's brain can differentiate into dopaminergic neurons, most dopaminergic neurons still aggregate in the neurospheres and fail to migrate out from the neurospheres. Thus, a new neural network cannot be established, and the therapeutic effect of dopaminergic progenitor cell transplantation is still limited.

In view of the above issues, inventors of the present invention found that in the differentiation progress of dopaminergic progenitor cells into dopaminergic neurons, adding BP to the culture environment of cells can induce the migration of dopaminergic neurons, promote the migration of dopaminergic neurons out from the neurospheres, and help establish neural connections to enhance the therapeutic effect of dopaminergic progenitor cell transplantation.

### SUMMARY OF THE INVENTION

An objective of the present invention is to provide a method for enhancing the therapeutic effect of dopaminergic progenitor cell transplantation, comprising culturing dopaminergic progenitor cells in a dopaminergic progenitor cell culture medium containing an active ingredient prior to transplanting the cells, wherein the active ingredient is BP and/or a pharmaceutically acceptable salt of BP. Preferably, the amount of the active ingredient in the culture medium ranges from about 0.5 μg (as BP) to about 20 μg (as BP) per mL of the culture medium. For example, the aforesaid method can enhance the therapeutic effect of dopaminergic progenitor cell transplantation on Parkinson's disease.

Another objective of the present invention is to provide a combination, comprising: (1) a conditional medium, comprising a basic medium and a neural induction factor; and (2) BP and/or a pharmaceutically acceptable salt of BP. In the aforesaid combination, the neural induction factor is at least one of a fibroblast growth factor, a transforming growth factor inhibitor, a glycogen synthase kinase inhibitor, and Purmorphamine, and wherein, the fibroblast growth factor is at least one of fibroblast growth factor-2 (FGF-2) and fibroblast growth factor-8b (FGF-8b), the transforming growth factor inhibitor is SB-431542, and the glycogen synthase kinase inhibitor is BIO. Preferably, the neural induction factor is at least one of FGF-8b and Purmorphamine.

Still another objective of the present invention is to provide a use of an active ingredient in the manufacture of a pharmaceutical composition, wherein the active ingredient is BP and/or a pharmaceutically acceptable salt of BP, and the pharmaceutical composition is administered in combination with dopaminergic progenitor cells pre-treated with BP and/or a pharmaceutically acceptable salt of BP in cell transplantation. The treatment of dopaminergic progenitor cells is conducted in a dopaminergic progenitor cell culture medium containing BP and/or a pharmaceutically acceptable salt of BP. Preferably, the amount of BP and/or a pharmaceutically acceptable salt of BP in the culture medium ranges from about 0.5 μg (as BP) to about 20 μg (as BP) per mL of the culture medium. Preferably, the pharmaceutical composition is provided in a form for oral administration, nasal administration, corticospinal injection, intrathecal injection, intracerebral injection, intravenous injection, peritoneal injection, or subcutaneous injection. Preferably, the dopaminergic progenitor cells are provided in a form for corticospinal injection, intrathecal injection, intracerebral injection, intravenous injection, peritoneal injection, or subcutaneous injection. For example, the aforesaid pharmaceutical composition is administered in combination with the dopaminergic progenitor cells pre-treated with BP and/or a pharmaceutically acceptable salt of BP in cell transplantation for treating Parkinson's disease.

Yet another objective of the present invention is to provide a pharmaceutical composition for cell transplantation, which comprises BP and/or a pharmaceutically acceptable salt of BP and is administered in combination with the dopaminergic progenitor cells pre-treated with BP and/or a pharmaceutically acceptable salt of BP. The treatment of dopaminergic progenitor cells is conducted in a dopaminergic progenitor cell culture medium containing BP and/or a pharmaceutically acceptable salt of BP. Preferably, the amount of BP and/or a pharmaceutically acceptable salt of BP in the culture medium ranges from about 0.5 μg (as BP) to about 20 μg (as BP) per mL of the culture medium. Preferably, the pharmaceutical composition is provided in a form for oral administration, nasal administration, corticospinal injection, intrathecal injection, intracerebral injection, intravenous injection, peritoneal injection, or subcutaneous injection. Preferably, the dopaminergic progenitor cells are provided in a form for corticospinal injection, intrathecal injection, intracerebral injection, intravenous injection, peritoneal injection, or subcutaneous injection. For example, the pharmaceutical composition is used in cell transplantation for treating Parkinson's disease.

Yet another objective of the present invention is to provide a method of cell transplantation, comprising separately or simultaneously administering to a subject in need an effective amount of dopaminergic progenitor cells and an effective amount of an active ingredient, wherein the dopaminergic progenitor cells are pre-treated with BP and/or a pharmaceutically acceptable salt of BP, and the active ingredient is BP and/or a pharmaceutically acceptable salt of BP. Preferably, the treatment of dopaminergic progenitor cells is conducted in a dopaminergic progenitor cell culture medium containing BP and/or a pharmaceutically acceptable salt of BP at an amount ranging from about 0.5 μg (as BP) to about 20 μg (as BP) per mL of the culture medium. Preferably, the active ingredient is administered to the subject by at least one of oral administration, nasal administration, corticospinal injection, intrathecal injection, intracerebral injection, intravenous injection, peritoneal injection, and subcutaneous injection. Preferably, the dopaminergic progenitor cells are administered to the subject by at least one of corticospinal injection, intrathecal injection, intracerebral injection, intravenous injection, peritoneal injection, and subcutaneous injection. For example, the aforesaid method is for treating Parkinson's disease.

The detailed technology and some particular embodiments implemented for the present invention are described in the following paragraphs for people skilled in this field to well appreciate the features of the claimed invention.

### BRIEF DESCRIPTION OF THE DRAWINGS

The patent application contains at least one drawing executed in color. Copies of this patent with color drawing(s) will be provided by the Patent and Trademark Office upon request and payment of the necessary fee.

FIG. 1 shows the result of using an Influx cell sorter to detect the expression of Corin on the cell surface.

FIG. 2 shows the pictures taken from a JuLI™Br cell imaging analyzer, and the pictures show the differentiation process of dopaminergic progenitor cells without BP treatment.

FIG. 3 shows the pictures taken from an inverted microscope on the dopaminergic progenitor cells in each group, wherein the cells in control group were cultured in a conditional medium free of BP for six days, and those in “BP (5)” group, “BP (10)” group, “BP (20)” group, “BP (50)” group, and “BP (100)” group were cultured in a conditional medium containing BP at the concentrations of 5, 10, 20, 50 and 100 μM, respectively, for six days.

FIG. 4 shows the pictures taken from a fluorescent microscope on the dopaminergic progenitor cells in each group, wherein the cells in control group were cultured in a conditional medium free of BP for ten days, and those in “BP (5)” group, “BP (10)” group, “BP (20)” group, “BP (50)” group, and “BP (100)” group were cultured in a conditional medium containing BP at the concentrations of 5, 10, 20, 50 and 100 μM, respectively, for ten days, and wherein the green, red, and blue fluorescence represents dopaminergic progenitor cells, dopaminergic neurons, and nuclei, respectively.

### DESCRIPTION OF THE PREFERRED EMBODIMENTS

The following will describe some of the embodiments of the present invention in detail. However, without departing from the spirit of the present invention, the present invention may be embodied in various embodiments and should not be limited to the embodiments described in the specification. In addition, unless otherwise indicated herein, the expressions “a,” “an,” “the,” or the like recited in the specification of the present invention (especially in the claims) are intended to include both the singular and plural forms. The term “pre-treat” or “pre-treating” used in this specification refers to the cells used for transplantation are treated with n-butylidenephthalide (BP) and/or a pharmaceutically acceptable salt of BP prior to conducting the cell transplantation. The term “subject” used in this specification refers to a mammalian, including human and non-human animals.

The numerical ranges (e.g., 5 to 100) used in this specification should be construed as including all of the rational numbers in the ranges and ranges consisting of any rational numbers in the ranges. Therefore, the numerical ranges used in this specification should include all the possible combinations of numerical values between the lowest value and the highest value listed therein.

The phrase “pharmaceutically acceptable salt” used in this specification includes “pharmaceutically acceptable base-addition salt” formed from “the above-mentioned compound containing functional acid group(s)” and “an organic or inorganic base”, and “pharmaceutically acceptable acid-addition salt” formed from “the above-mentioned compound containing functional base group(s)” and “an organic or inorganic acid”.

Examples of the “pharmaceutically acceptable base-addition salts” formed with inorganic bases include, but are not limited to, alkali metal salts (e.g., sodium salts and potassium salts), alkaline-earth metal salts (e.g., calcium salts and magnesium salts), transition metal salts (e.g., ferric salts, zinc salts, copper salts, manganese salts and aluminum salts) and ammonium salts.

Examples of the “pharmaceutically acceptable base-addition salts” formed with organic bases include, but are not limited to, salts formed with methylamine, dimethylamine, trimethylamine, ethylamine, diethylamine, triethylamine, isopropylamine, tripropylamine, tributylamine, ethanolamine, diethanolamine, 2-dimethylaminoethanol, 2-diethylaminoethanol, dicyclohexylamine, lysine, arginine, histidine, caffeine, hydrabamine, choline, betaine, ethylenediamine, glucosamine, methylglucamine, theobromine, purine, piperidine, N-ethylpiperidine, tetramethylammonium compound, tetraethylammonium compound, pyridine, N,N-dimethylaniline, N-methylpiperidine, N-methylmorpholine, dicyclohexylamine, dibenzyl amine, N,N-dibenzylphenethylamine, I-ephenamine, N,N-dibenzylethylenediamine, polyamine resin and its analogs, etc.

Examples of the “pharmaceutically acceptable acid-addition salts” formed with inorganic acids include, but are not limited to, salts formed with hydrobromic acid, hydrochloric acid, sulfuric acid, nitric acid, phosphoric acid, hyperchloric acid, etc.

Examples of the “pharmaceutically acceptable acid-addition salts” formed with organic acids include, but are not limited to, salts formed with sulfonic acid (e.g., p-toluenesulfonic acid, benzenesulfonic acid, methanesulfonic acid, ethylsulfonic acid, 2-hydroxyethanesulfonic acid and naphthalenesulfonic acid), carboxylic acid (e.g., acetic acid, propionic acid, fumaric acid, maleic acid, benzoic acid, salicylic acid and succinic acid), anionic amino acid (e.g., glutamic acid and aspartic acid), hydroxy acid (e.g., citric acid, lactic acid, tartaric acid, glycolic acid and malic acid), fatty acid (e.g., hexanoic acid, octanoic acid, decanoic acid, oleic acid and stearic acid), dihydroxynaphthoic acid, resinolic acid, etc.

The term “as n-butylidenephthalide (BP)” used in this specification refers to when the pharmaceutically acceptable salt of BP is used as an active ingredient, the amount of BP that can be provided by the pharmaceutically acceptable salt.

As described above, the cause of Parkinson's disease primarily resides in the decreased dopamine secretion due to the degeneration and/or death of dopaminergic neurons. Patients with Parkinson's disease will lose their motor control ability gradually. Dopaminergic progenitor cell transplantation has brought new opportunities in treating Parkinson's disease. Currently, dopaminergic progenitor cells used for cell transplantation are mostly differentiated from inducing embryonic stem cells. For example, culturing embryonic stem cells in a basic medium added with neural induction factors such as a fibroblast growth factor (e.g., FGF-2, FGF-8b), a transforming growth factor inhibitor (e.g., SB-431542), a glycogen synthase kinase inhibitor (e.g., BIO), and Purmorphamine, can induce the differentiation of embryonic stem cells into dopaminergic progenitor cells.

However, when dopaminergic progenitor cells are transplanted as neurospheres to a patient's brain, though they can differentiate into dopaminergic neurons, most dopaminergic neurons still aggregate in the neurospheres and fail to migrate out from the neurospheres. Thus, a new neural network cannot be established, and the therapeutic effect is limited. These can be noted in “Human iPS cell-derived dopaminergic neurons function in a primate Parkinson's disease model. Nature 548, 592-596 (2017)” and “Predictive Markers Guide Differentiation to Improve Graft Outcome in Clinical Translation of hESC-Based Therapy for Parkinson's Disease. Cell stem cell 20, 135-148, (2017),” which are entirely incorporated hereinto by reference.

Inventors of the present invention discovered that in the differentiation process of dopaminergic progenitor cells into dopaminergic neurons, adding BP to the culture environment can induce the migration of dopaminergic neurons, promote the migration of dopaminergic neurons out from the neurospheres, and help establish neural connections. Thus, BP can be used in dopaminergic progenitor cell transplantation to promote the migration of dopaminergic neurons out from the neurospheres after dopaminergic progenitor cells differentiate into dopaminergic neurons and help establish neural connections, thereby enhancing the therapeutic effect of dopaminergic progenitor cell transplantation.

The present invention relates to the effect of BP in enhancing the therapeutic effect of dopaminergic progenitor cell transplantation and uses of the same. The present invention especially relates to the effect of BP in enhancing the therapeutic effect of dopaminergic progenitor cell transplantation on Parkinson's disease. The uses include providing a method and a combination for enhancing the therapeutic effect of dopaminergic progenitor cell transplantation. The method comprises culturing dopaminergic progenitor cells in a dopaminergic progenitor cell culture medium containing BP and/or a pharmaceutically acceptable salt of BP prior to transplanting the cells, and the combination comprises: (1) a conditional medium, comprising a basic medium and a neural induction factor, and (2) BP and/or a pharmaceutically acceptable salt of BP.

In the method for enhancing the therapeutic effect of dopaminergic progenitor cell transplantation in accordance with the present invention, the phrase “pre-treating the dopaminergic progenitor cells by culturing the dopaminergic progenitor cells in a dopaminergic progenitor cell culture medium containing BP and/or a pharmaceutically acceptable salt of BP” means that when conducting the treatment, the dopaminergic progenitor cells were placed in the culture medium. In addition, the dopaminergic progenitor cell culture medium used in the method in accordance with the present invention comprises a basic medium and a neural induction factor, wherein the basic medium comprises the essential ingredients capable of providing nutrient and condition (e.g., pH value) for dopaminergic progenitor cell growth. In general, examples of the basic medium include, but are not limited to, a DMEM/F12 medium (Dulbecco's Modified Eagle Medium: Nutrient Mixture F-12) that is externally added with N2 supplement, and a neural basal medium that is externally added with N2 supplement. For example, the DMEM/F12 medium that is externally added with N2 supplement can be used as a basic medium to conduct the treatment of dopaminergic progenitor cells.

In the method in accordance with the present invention, the amount of BP and/or a pharmaceutically acceptable salt of BP used in the culture medium to treat the dopaminergic progenitor cells usually ranges from about 0.5 μg (as BP) to about 20 μg (as BP) per mL of the culture medium, preferably ranges from about 2 μg (as BP) to about 15 μg (as BP) per mL of the culture medium, and more preferably ranges from about 3 μg (as BP) to about 12 μg (as BP) per mL of the culture medium. For example, as shown by the appended examples, BP can effectively induce the migration of dopaminergic neurons and promote the migration of dopaminergic neurons out from the neurospheres at an amount ranging from about 0.9 μg (as BP) to about 19 μg (as BP) per mL of the culture medium (i.e., the amount of BP is ranging from about 5 μM to about 100 M).

The combination provided in accordance with the present invention comprises: (1) a conditional medium, comprising a basic medium and a neural induction factor, and (2) BP and/or a pharmaceutically acceptable salt of BP. In the combination, the applied type of the basic medium and the used amount of BP and/or a pharmaceutically acceptable salt of BP are all in line with the above descriptions.

Besides the basic medium, the component (1) (i.e., a conditional medium) of the combination provided in accordance with the present invention could further comprise any neural induction factor that is capable of helping induce the differentiation of stem cells into dopaminergic progenitor cells, such as a fibroblast growth factor, a transforming growth factor inhibitor, a glycogen synthase kinase inhibitor, Purmorphamine, or a combination thereof, but is not limited thereby. Preferably, the fibroblast growth factor is at least one of fibroblast growth factor-2 (FGF-2) and fibroblast growth factor-8b (FGF-8b), the transforming growth factor inhibitor is SB-431542, and the glycogen synthase kinase inhibitor is BIO. More preferably, the neural induction factor is at least one of fibroblast growth factor-8b (FGF-8b) and Purmorphamine.

The combination provided in accordance with the present invention could be a kit or a composition. When the combination is a kit, the component (1) (i.e., a conditional medium) and component (2) (i.e., BP and/or a pharmaceutically acceptable salt of BP) are normally independently packaged and stored in different containers (e.g., a plastic bag, a plastic bottle, a glass bottle, an ampoule), and could be transported and sold separately or in a set. Optionally, the subcomponent of the component (1) could also be independently packaged and stored. The kit could further comprise an instruction manual, which provides the procedures and programs for the user to mix the components on-site for culturing, treating and using the cells.

For example, when the subcomponents of component (1) and component (2) are independently packaged and stored and transported or sold separately, the neural induction factor(s) (e.g., FGF-2, FGF-8b, SB-431542 and BIO) and BP and/or a pharmaceutically acceptable salt of BP could be kept in a dark environment at a temperature of less than −20□, and the basic medium could be kept in an environment at −20. Also, when the components in the kit in accordance with the present invention are transported and sold in a set, the neural induction factor(s) and BP and/or a pharmaceutically acceptable salt of BP could be kept in a container with an interior temperature of −20, and the basic medium could be kept in a container with an interior temperature of −20. There is no particular limitation on the shape and size of the containers, as long as the containers could serve the desired insulation function to ensure that the storage temperatures of components will not affect each other when the components are transported and sold in a set.

When using the kit in accordance with the present invention, there is no particular limitation for the order to mix each component. For example, when the subcomponents of the conditional medium are independently packaged, the conditional medium could be formulated first, and then the conditional medium is mixed with BP and/or a pharmaceutically acceptable salt of BP. Also, the basic medium could be mixed with BP and/or a pharmaceutically acceptable salt of BP to provide a mixture, and then the mixture is mixed with other subcomponents. Alternatively, all the subcomponent of the conditional medium and BP and/or a pharmaceutically acceptable salt of BP could be mixed simultaneously. In addition, BP and/or a pharmaceutically acceptable salt of BP could be directly mixed with the conditional medium or basic medium; or BP and/or a pharmaceutically acceptable salt of BP could be dissolved in a solvent to provide a BP solution, and then the BP solution is mixed with the conditional medium or basic medium. Examples of the solvent that is capable of dissolving BP and/or a pharmaceutically acceptable salt of BP include, but are not limited to, dimethyl sulfoxide (DMSO), ethanol and vegetable oil.

When the combination provided in accordance with the present invention is a composition, the component (i) and component (2) are normally mixed and stored together in a container (e.g., a plastic bag, a plastic bottle, a glass bottle, an ampoule).

According to the present invention, the use of the combination described above in dopaminergic progenitor cell transplantation can induce the migration of dopaminergic neurons, promote the migration of dopaminergic neurons out from the neurospheres, and help establish neural connections, thereby enhancing the therapeutic effect of dopaminergic progenitor cell transplantation. For example, when using the combination of the present invention in dopaminergic progenitor cell transplantation, stem cells are cultured in a conditional medium comprising a basic medium and the neural induction factor(s) (e.g., FGF-2, FGF-8b, SB-431542, BIO and Purmorphamine) to induce the differentiation of the stem cells into dopaminergic progenitor cells; then, the aforesaid conditional medium is replaced with another conditional medium comprising a basic medium and BP and/or a pharmaceutically acceptable salt of BP to continuously culture the dopaminergic progenitor cells for about eight to twelve days; finally, the dopaminergic progenitor cells thus provided are transplanted to a subject in need.

In the use of BP and/or a pharmaceutically acceptable salt of BP in the manufacture of a pharmaceutical composition in accordance with the present invention, the pharmaceutical composition is administered in combination with the dopaminergic progenitor cells pre-treated with BP and/or a pharmaceutically acceptable salt of BP in cell transplantation. The treatment of dopaminergic progenitor cells is conducted in a dopaminergic progenitor cell culture medium containing BP and/or a pharmaceutically acceptable salt of BP at an amount ranging from about 0.5 μg (as BP) to about 20 μg (as BP) per mL of the culture medium, preferably ranging from about 2 μg (as BP) to about 15 μg (as BP) per mL of the culture medium, and more preferably ranging from about 3 μg (as BP) to about 12 μg (as BP) per mL of the culture medium.

Depending on the desired purpose(s), the pharmaceutical composition of the present invention could be provided in any suitable form without particular limitations. For example, the pharmaceutical composition could be administered to a subject in need by an oral or parenteral (e.g., nasal administration, corticospinal injection, intrathecal injection, intracerebral injection, intravenous injection, peritoneal injection, and subcutaneous injection) route, but is not limited thereby. Depending on the form and purpose(s), a suitable carrier could be chosen and used to provide the pharmaceutical composition, wherein examples of the carrier include excipients, diluents, auxiliaries, stabilizers, absorbent retarders, disintegrating agent, hydrotropic agents, emulsifiers, antioxidants, adhesives, binders, tackifiers, dispersants, suspending agents, lubricants, hygroscopic agents, etc.

As a form for oral administration, the pharmaceutical composition could comprise any pharmaceutically acceptable carrier that will not adversely affect the desired effects of the active ingredient (i.e., BP and/or a pharmaceutically acceptable salt of BP). Examples of the suitable carrier include, but are not limited to, water, saline, dextrose, glycerol, ethanol or its analogs, cellulose, starch, sugar bentonite, and combinations thereof. The pharmaceutical composition could be provided by any suitable method in any suitable form for oral administration, such as in a form of a tablet (e.g., sugar-coated tablet), a pill, a capsule, granules, a pulvis, a fluidextract, a solution, syrup, a suspension, a tincture, etc.

As for the form of injections or drips suitable for corticospinal administration, intrathecal administration, intracerebral administration, intravenous administration, peritoneal administration, or subcutaneous administration, the pharmaceutical composition could comprise one or more ingredient(s), such as an isotonic solution, a salt-buffered saline (e.g., phosphate-buffered saline or citrate-buffered saline), a hydrotropic agent, an emulsifier, a 5% sugar solution, and other carriers to provide the pharmaceutical composition as an intravenous infusion, an emulsified intravenous infusion, a powder for injection, a suspension for injection, or a powder suspension for injection, etc. Alternatively, the pharmaceutical composition could be prepared as a pre-injection solid. The pre-injection solid could be provided in a form which is soluble in other solutions or suspensions, or in an emulsifiable form. A desired injection is provided by dissolving the pre-injection solid in other solutions or suspensions or emulsifying it prior to being administered to the subject in need.

Optionally, the pharmaceutical composition provided in accordance with the present invention could further comprise a suitable amount of additives, such as a flavoring agent, a toner, or a coloring agent for enhancing the palatability and the visual perception of the pharmaceutical composition, and/or a buffer, a conservative, a preservative, an antibacterial agent, or an antifungal agent for improving the stability and storability of the pharmaceutical composition. Optionally, the pharmaceutical composition could further comprise one or more other active ingredients, or be used in combination with a medicament comprising one or more other active ingredients, to further enhance the effect of the pharmaceutical composition, or to increase the application flexibility and adaptability of the preparation thus provided, as long as the other active ingredients do not adversely affect the desired effects of the active ingredient of the present invention (i.e., BP and/or a pharmaceutically acceptable salt of BP).

In the use in accordance with the present invention, in addition to the pharmaceutical composition provided in accordance with the present invention, the dopaminergic progenitor cells pre-treated with BP and/or a pharmaceutically acceptable salt of BP should also be administered to the subject in need, and the dopaminergic progenitor cells and the pharmaceutical composition could be administered simultaneously or separately. Depending on the desired purpose(s), the pre-treated dopaminergic progenitor cells could be administered via any suitable route without particular limitations. For example, the pre-treated dopaminergic progenitor cells could be provided in a form suitable for injection or cell infusion and could be administered to the subject by corticospinal injection, intrathecal injection, intracerebral injection, intravenous injection, peritoneal injection, or subcutaneous injection, but is not limited thereby. One or more pharmaceutical acceptable carriers (e.g., a normal saline) could be used to provide the pre-treated dopaminergic progenitor cells in a form suitable for injection or cell infusion.

Depending on the needs, age, body weight and health conditions of the subject, the pharmaceutical composition and the dopaminergic progenitor cells pre-treated with BP and/or a pharmaceutically acceptable salt of BP could be administered separately at various administration frequencies, such as once a day, multiple times a day, once every few days, etc. In addition, the concentration of the active ingredient (i.e., BP and/or a pharmaceutically acceptable salt of BP) in the pharmaceutical composition provided in accordance with the present invention could be adjusted depending on the requirements of practical application. For example, when the pharmaceutical composition is administered to a subject by oral administration twice a day, and the dopaminergic progenitor cells pre-treated with BP and/or a pharmaceutically acceptable salt of BP are administered to the subject by intracerebral injection once every two weeks to treat and/or delay the onset of Parkinson's disease, the pharmaceutical composition is usually administered at an amount ranging from about 30 mg (as BP)/kg-body weight to about 2000 mg (as BP)/kg-body weight every time, preferably ranging from about 50 mg (as BP)/kg-body weight to about 1000 mg (as BP)/kg-body weight every time, and more preferably ranging from about 100 mg (as BP)/kg-body weight to about 500 mg (as BP)/kg-body weight every time. The unit “mg/kg-body weight” refers to the amount required for per kg-body weight of the subject. Furthermore, the dopaminergic progenitor cells are usually administered at an amount ranging from about 1×10⁵ cells to about 5×10⁶ cells, and preferably ranging from about 1×10⁶ cells to about 2×10⁶ cells.

The present invention also relates to a method of cell transplantation, comprising separately or simultaneously administering to a subject in need an effective amount of dopaminergic progenitor cells and an effective amount of an active ingredient, wherein the dopaminergic progenitor cells are pre-treated with BP and/or a pharmaceutically acceptable salt of BP, and the active ingredient is BP and/or a pharmaceutically acceptable salt of BP. The term “a subject in need” refers to a subject suffering from dopaminergic neuron degeneration, dopaminergic neuron death, and/or insufficient secretion of dopamine. In the method of cell transplantation of the present invention, the treatment of the dopaminergic progenitor cells, and the administration types of the pre-treated dopaminergic progenitor cells and active ingredient are all in line with the above descriptions.

The present invention will be further illustrated in detail with specific examples as follows. However, the following examples are provided only for illustrating the present invention and the scope of the present invention is not limited thereby. The scope of the present invention will be indicated in the appended claims.

### EXAMPLES

### Preparation Examples

A. Preparation of a Conditional Medium

A-1.

A DMEM/F12 medium (Dulbecco's Modified Eagle Medium/Nutrient Mixture F-12; purchased from Gibco company, product number: 11320033), externally added with N2 supplement (purchased from Gibco company, product number: 17502048), was used as a basic medium and further added with the neural induction factors including BIO (purchased from Sigma-Aldrich company, product number: B1686), SB-431542 (purchased from Sigma-Aldrich company, product number: S4317), FGF-2 (purchased from Peprotech company, product number: 100-18B), Purmorphamine (purchased from Cayman Chemical company, product number: 10009634), and FGF-8b (purchased from R&D System company, product number: 423-F8) to the final concentrations of 0.5 μM BIO, 10 μM SB-431542, 10 ng/mL FGF-2, 1 μM Purmorphamine and 50 ng/mL FGF-8b, respectively, so as to provide a conditional medium.

A-2.

Another conditional medium was provided in accordance with [Preparation Example A-1], but the neural induction factors added to the basic medium only include Purmorphamine and FGF-8b, and the final concentrations of Purmorphamine and FGF-8b were 1 μM and 50 ng/mL, respectively.

B. Preparation of Dopaminergic Progenitor Cells

B-1. Pre-Culture of Embryonic Stem Cells

Embryonic stem cells (provided by Lee Women's Hospital, Taiwan) were cultured in a DMEM/F12 medium containing 20% KnockOut Serum Replacement (KSR; purchased from Gibco company, product number: 10828028) for two days, so as to become the suspended globular cells.

B-2. Differentiation of Embryonic Stem Cells

The suspended globular embryonic stem cells provided by [Preparation Example B-1] were cultured in the conditional medium provided by [Preparation Example A-1] for two days. Then, the medium was removed, and the cells were continuously cultured in the conditional medium provided by [Preparation Example A-2] for six days to obtain a cell liquid.

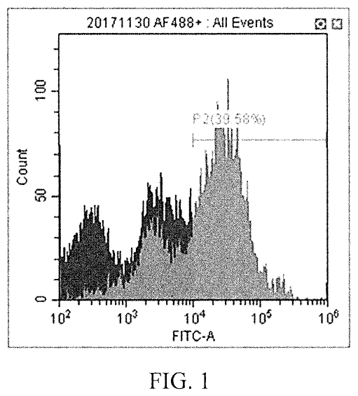

It is known that Corin is a specific surface protein of ventral midbrain. Thus, Corin antibody (purchased from R&D System company, product number: MAB2209) was added to the cell liquid obtained above and incubated for 15 minutes. The cells were washed with PBS once. Then, a second antibody with fluorescence (purchased from Invitrogen company, product number: A21208) was added thereinto and incubated for 15 minutes. The cells were washed with PBS once. Finally, the cells were suspended in PBS. The fluorescence of the aforesaid cell suspension was detected by an Influx cell sorter (purchased from BD company) (as shown in FIG. 1, there are 39.6% of cells expressing Corin), and the cells with fluorescent signals (i.e., the dopaminergic progenitor cells of ventral midbrain) were sorted.

C. Treatment of Dopaminergic Progenitor Cells

The dopaminergic progenitor cells provided by [Preparation Example B-2] were cultured in the conditional medium provided by [Preparation Example A-2] at 37, 5% CO₂ for 24 hours. Then, the cells were divided into six groups and independently subjected to the following treatments:
(1) Control group: cells were continuously cultured in the conditional medium provided by [Preparation Example A-2] (i.e., the medium free of BP) for ten days.
(2) “BP (5)” group, “BP (10)” group, “BP (20)” group, “BP (50)” group, and “BP (100)” group: cells were cultured in accordance with the condition of control group, but the conditional medium was further added with BP (purchased from Sigma-Aldrich company, product number: W333301) at final concentrations of 5, 10, 20, 50, and 100 μM, respectively.

### Example 1: Influence of n-Butylidenephthalide (BP) on the Differentiation Ability of Dopaminergic Progenitor Cells

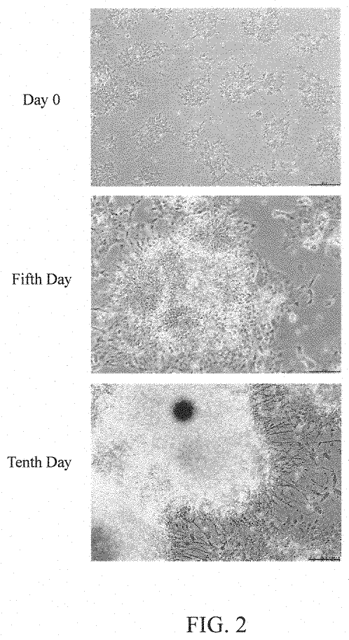

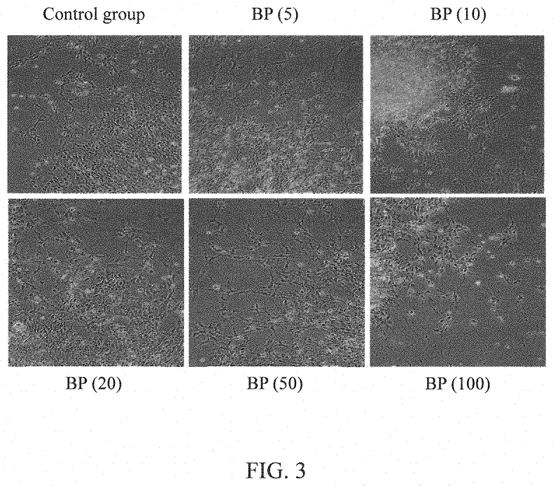

To understand the influence of BP on the differentiation of dopaminergic progenitor cells into dopaminergic neurons, the differentiation process of the cells in control group provided by [Preparation Example C] was continuously taken from a JuLI™Br cell imaging analyzer (purchased from NanoEnTek company) (results are shown in FIG. 2), and the cell morphology in each group provided by [Preparation Example C] was taken from and recorded by an inverted microscope (purchased from Nikon company) when the cells were continuously cultured for six days (results are shown in FIG. 3).

As shown in FIG. 2, when the cells in control group (i.e., the dopaminergic progenitor cells untreated with BP) were continuously cultured for five days, the phenomenon of neural differentiation can be observed, and when the cells were continuously cultured for ten days, the cells have differentiated into the dopaminergic neurons with axon pattern. As shown in FIG. 3, the formation of nerve fibers can be observed in all the control group, “BP (5)” group, “BP (10)” group, “BP (20)” group, “BP (50)” group, and “BP (100)” group. These results indicate that the treatment of BP does not influence the normal neural differentiation of dopaminergic progenitor cells.

### Example 2: Influence of n-Butylidenephthalide (BP) on Promoting the Migration of Dopaminergic Neurons

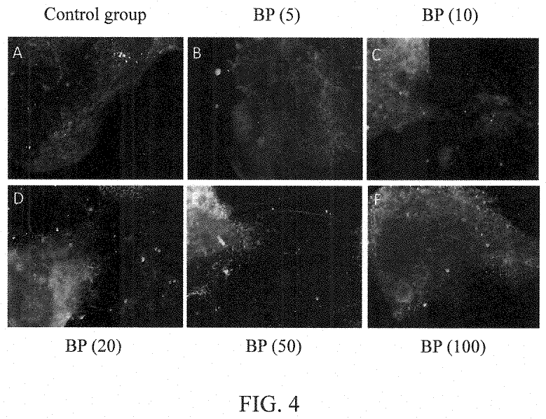

To understand the influence of BP on dopaminergic neurons, when the cells in each group provided by [Preparation Example C] were continuously cultured for ten days, the cells were fixed and then subjected to fluorescent staining with the antibodies including Sox-1 (purchased from Santa Cruz company, product number: SC-17318), TH (tyrosine hydroxylase; purchased from Millipore company, product number: MAB152) and DAPI (diamidino-2-phenylindole; purchased from ThermoFisher Scientific company, product number: D1306), respectively (Sox-1 expresses in dopaminergic progenitor cells, TH expresses in dopaminergic neurons, and DAPI is a nuclear-specific dye). Then, the dopaminergic progenitor cells (green fluorescence), dopaminergic neurons (red fluorescence) and nuclei (blue fluorescence) were observed by an upright fluorescent microscope (purchased from Nikon company). The results are shown in FIG. 4.

A shown in FIG. 4, as compared to control group, the phenomenon of dopaminergic neurons migrating out from the neurospheres can be observed in “BP (5)” group, “BP (10)” group, “BP (20)” group, “BP (50)” group, and “BP (100)” group, wherein the phenomenon in “BP (50)” group was most significant. These results indicate that BP indeed can induce the migration of dopaminergic neurons effectively, and thus, can be used in dopaminergic progenitor cell transplantation to promote the migration of dopaminergic neurons out from the neurospheres after they differentiated from dopaminergic progenitor cells, and help establish neural connections, thereby enhancing the therapeutic effect of dopaminergic progenitor cell transplantation.

What is claimed is:

1. A method for enhancing the therapeutic effect of dopaminergic progenitor cell transplantation, comprising culturing dopaminergic progenitor cells in a dopaminergic progenitor cell culture medium containing an active ingredient prior to transplanting the cells, wherein the active ingredient is n-butylidenephthalide (BP) and/or a pharmaceutically acceptable salt of BP.

2. The method as claimed in claim 1, wherein the amount of the active ingredient in the culture medium ranges from about 0.5 μg (as BP) to about 20 μg (as BP) per mL of the culture medium.

3. The method as claimed in claim 1, which is for enhancing the therapeutic effect of dopaminergic progenitor cell transplantation on Parkinson's disease.

4. The method as claimed in claim 2, which is for enhancing the therapeutic effect of dopaminergic progenitor cell transplantation on Parkinson's disease.

5. A combination, comprising:
(1) a conditional medium, comprising a basic medium and a neural induction factor; and
(2) n-butylidenephthalide (BP) and/or a pharmaceutically acceptable salt of BP.

6. The combination as claimed in claim 5, wherein the neural induction factor is at least one of a fibroblast growth factor, a transforming growth factor inhibitor, a glycogen synthase kinase inhibitor, and Purmorphamine, and wherein, the fibroblast growth factor is at least one of fibroblast growth factor-2 (FGF-2) and fibroblast growth factor-8b (FGF-8b), the transforming growth factor inhibitor is SB-431542, and the glycogen synthase kinase inhibitor is BIO.

7. The combination as claimed in claim 6, wherein the neural induction factor is at least one of fibroblast growth factor-8b (FGF-8b) and Purmorphamine.

8. A method of cell transplantation, comprising separately or simultaneously administering to a subject in need an effective amount of dopaminergic progenitor cells and an effective amount of an active ingredient, wherein the dopaminergic progenitor cells are pre-treated with n-butylidenephthalide (BP) and/or a pharmaceutically acceptable salt of BP, and the active ingredient is n-butylidenephthalide (BP) and/or a pharmaceutically acceptable salt of BP.

9. The method as claimed in claim 8, wherein the treatment of dopaminergic progenitor cells is conducted in a dopaminergic progenitor cell culture medium containing n-butylidenephthalide (BP) and/or a pharmaceutically acceptable salt of BP at an amount ranging from about 0.5 μg (as BP) to about 20 μg (as BP) per mL of the culture medium.

10. The method as claimed in claim 8, which is for treating Parkinson's disease.

11. The method as claimed in claim 8, wherein the active ingredient is administered to the subject by oral administration, nasal administration, corticospinal injection, intrathecal injection, intracerebral injection, intravenous injection, peritoneal injection, subcutaneous injection, or a combination thereof.

12. The method as claimed in claim 8, wherein the dopaminergic progenitor cells are administered to the subject by corticospinal injection, intrathecal injection, intracerebral injection, intravenous injection, peritoneal injection, subcutaneous injection, or a combination thereof.

13. The method as claimed in claim 9, which is for treating Parkinson's disease.

14. The method as claimed in claim 9, wherein the active ingredient is administered to the subject by oral administration, nasal administration, corticospinal injection, intrathecal injection, intracerebral injection, intravenous injection, peritoneal injection, subcutaneous injection, or a combination thereof.

15. The method as claimed in claim 9, wherein the dopaminergic progenitor cells are administered to the subject by corticospinal injection, intrathecal injection, intracerebral injection, intravenous injection, peritoneal injection, subcutaneous injection, or a combination thereof.
